(19) United States
(12) Patent Application Publication
(10) Pub. No.: US 2012/0302740 A1
(43) Pub. Date: Nov. 29, 2012

(54) VINCRISTINE IMMUNOASSAY

(75) Inventors: Salvatore J. Salamone, Stockton, NJ (US); Jodi Blake Courtney, Doylestown, NJ (US); Daniel J. Cline, Allentown, PA (US)

(73) Assignee: SALADAX BIOMEDICAL INC., Bethlehem, PA (US)

(21) Appl. No.: 13/569,431

(22) Filed: Aug. 8, 2012

Related U.S. Application Data
(62) Division of application No. 12/900,923, filed on Oct. 8, 2010.

(51) Int. Cl. C07K 16/44; C07D 487/18

(52) U.S. Cl. 530/388.9; 530/389.8; 540/478

(57) ABSTRACT

Novel conjugates and immunogens derived from vincristine and antibodies generated by these immunogens are useful in immunoassays for the quantification and monitoring of vincristine in biological fluids.

### CROSS-REFERENCE TO RELATED APPLICATION

This application is a divisional of Ser. No. 12/900,923, filed Oct. 8, 2010, entitled “Vincristine Immunoassay”. This application is hereby incorporated by reference herein in its entirety.

### FIELD OF THE INVENTION

This invention relates to the field of immunological assays for determining the presence and/or quantifying the amount of active vincristine in human biological samples in order to rapidly determine optimal drug concentrations during chemotherapy.

### BACKGROUND OF THE INVENTION

Cancer is a term used to describe a group of malignancies that all share the common trait of developing when cells in a part of the body begin to grow out of control. Most cancers form as tumors, but can also manifest in the blood and circulate through other tissues where they grow. Cancer malignancies are most commonly treated with a combination of surgery, chemotherapy, and/or radiation therapy. The type of therapy used to treat a specific cancer depends upon several factors including the type of cancer malignancy and the stage during which it was diagnosed.

One particularly potent class of chemotherapeutic agent was originally isolated from the periwinkle plant, the vinca alkaloids. This class of alkaloid targets microtubules involved in spindle formation, thereby arresting cell division and causing the rapidly dividing cancer cells (neoplasia) to die. Included in the vinca alkaloids are vinblastine, vinrelbine, vindesine, and vincristine. The chemical structures of these alkaloids are very similar, varying mostly by only one hydroxyl group, and yet each has differing anti-neoplastic efficacies and toxicities. The structures of vincristine (I) and related vinca alkaloids vinblastine (IIA), vindesine (IIB), and vinorelbine (IIC) are shown below.

Vincristine has the following formula:

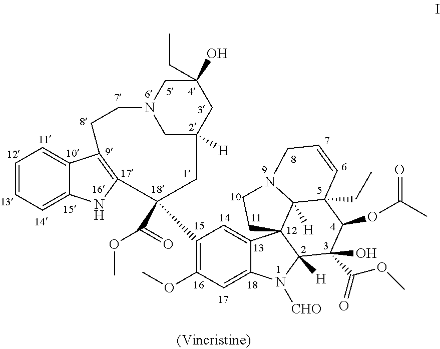

Related alkaloids have the following formulae:

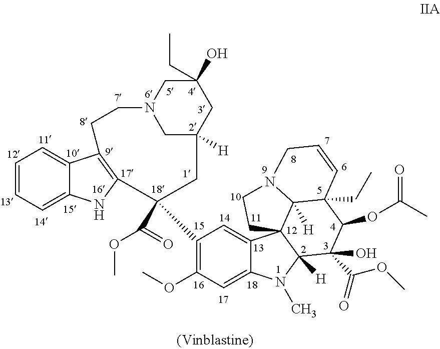

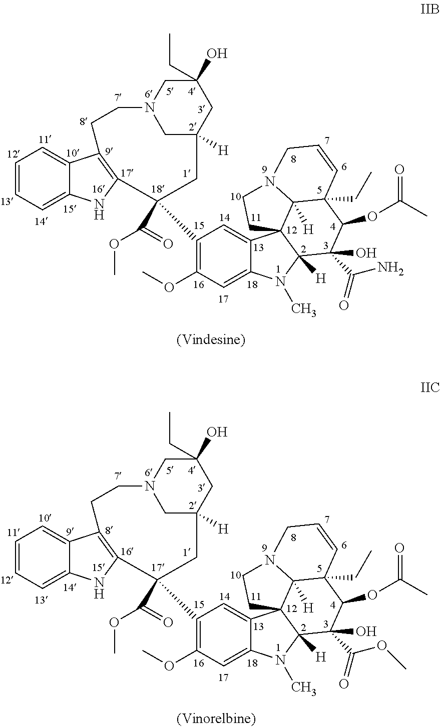

Of the vinca alkaloids, vincristine is used in the most chemotherapeutic regimens across a wide range of cancer types. The primary indications for vincristine are most types of leukemia, particularly (acute and/or chronic) myelogenous and lymphocytic leukemias, but also Hodgkins and non-Hodgkins lymphomas, Wilms' Tumor, neuroblastoma, and rhabdomyosarcoma (Package-Insert-Vincristine, 1999). Vincristine, also known as leurocristine, is marketed under several names: Oncovin® by Eli Lilly Pharmaceuticals or Vincasar PFS® by GensiaSicor Pharmaceuticals, or generic vincristine sulfate by Teva Pharmaceuticals.

Vincristine sulfate is given as a single one minute intravenous injection of 1-2 mg per m² body surface area. The drug is rapidly distributed into tissue within 10 minutes, and has an elimination half-life of 19-155 hours. This large variation has been traced to several factors including:
Age
Weight
Organ function
Drug-drug interaction
Genetic regulation
Ethnicity

Both inter-patient and intra-patient variability is seen, with up to 40-fold difference in total dose exposure (Crom et al., 1994, Journal of Pediatrics), and up to 25-fold interpatient variability in clearance (Gidding et al., 1999, Cancer Chemotherapy and Pharmacology; Groninger et al., 2002, Pediatric Research). This variability can impact efficacy and safety (Gidding et al., 1999, Critical Reviews in Oncology Hematology; Moore et al., 2009, Pediatric Blood & Cancer). Since efficacy of vincristine is improved at higher trough levels and that the drug exhibits wide intra- and inter-patient pharmacokinetic variability, monitoring concentrations of this drug in blood and adjusting to target levels would be of value in increasing efficacy and minimizing toxicity (Vanwarmerdam et al., 1995, Cancer Research Therapy & Control).

As a result of this variability, equal doses of the same drug in different individuals can result in dramatically different clinical outcomes. The effectiveness of the same dosage of vincristine varies significantly based upon individual drug clearance and the ultimate serum drug concentration in the patient (Lonnerholm et al., 2008, British Journal of Haematology). Therapeutic drug management would provide the clinician with insight on patient variation in drug administration. With therapeutic drug management, drug dosages could be individualized to the patient, and the chances of effectively treating the disorder without the unwanted side effects would be much higher.

Routine therapeutic drug management of vincristine would require the availability of simple automated tests adaptable to general laboratory equipment. A radioimmunoassay had been developed, but was limited in its usefulness as it used rabbit antiserum to vinblastine (IIA), and was therefore non-specific for vincristine (Sethi et al., 1980, Cancer Chemotherapy and Pharmacology). The use of liquid chromatography (LC) with UV or mass spectrometry detection to determine the concentration of vincristine in human blood and plasma has been described (Bloemhof et al., 1991, Journal of Chromatography-Biomedical Applications; Koopmans et al., 2001, Therapeutic Drug Monitoring; Damen et al., 2010, Biomedical Chromatography). These methods, while highly sensitive, are labor intensive, requiring liquid-liquid or solid phase extractions, use expensive equipment and are not amenable to routine clinical laboratory use.

As seen from the foregoing, there are no sensitive immunoassays specific for determining the presence and/or quantifying the amount of vincristine in human biological fluids. Routine therapeutic drug management of vincristine by immunoassays would provide simple automated tests adapted to standard laboratory equipment. However, in order to provide such immunoassays, antibodies specific to vincristine must be produced. The derivatives and immunogen used in this assay must impart through these corresponding antibodies specific reactivity to vincristine without any substantial cross reactivity to other therapeutically or pharmacologically inactive related vinca alkaloids. In order to be effective in monitoring drug levels, the antibodies should be specific to vincristine and not cross reactive with pharmaceutically or pharmacologically inactive metabolites of vincristine. The principle pharmaceutically or pharmacologically inactive metabolite of vincristine is ‘M1’ (Dennison et al., 2006, Drug Metabolism and Disposition; 34 (8), 1317-1327; and Dennison et al., 2008, Therapeutic Drug Monitoring)30 (3):357-361, which has the formula:

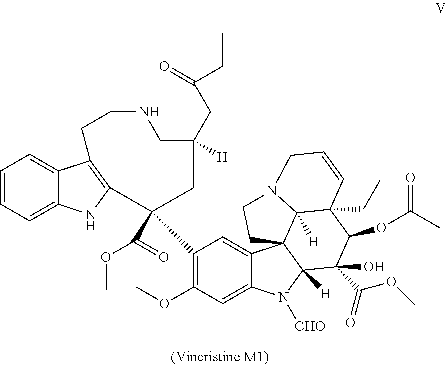

### SUMMARY OF INVENTION

In accordance with this invention, a new class of monoclonal antibodies have been produced which are substantially selectively reactive to vincristine so as to selectively bind to vincristine without any substantial cross reactivity to the related vinca alkaloids, such as vinblastine, vindesine, or vinorelbine, and to pharmacologically or pharmaceutically inactive metabolites of vincristine, particularly M1. By selectively reactivity, it is meant that this antibody only reacts to bind with the vincristine and does not substantially react with to bind to the pharmacologically active related vinca alkaloids, such as vinblastine, vindesine, or vinorelbine, or with the pharmacologically inactive vincristine metabolites, particularly M1. These properties are important for providing the immunoassay of this invention since the metabolite, M1, is produced from vincristine after administration to cancer patients undergoing chemotherapy. Therefore, in carrying out these immunoassays, on a human fluid sample, there may be, pharmacologically or pharmaceutically inactive metabolites of vincristine, particularly M1 in the human sample of the patient treated with vincristine. With this reactivity and cross reactivity of these antibodies to the non-pharmaceutically active metabolites of vincristine, such as M1 these metabolites will not interfere with an accurate determination, by an immunoassay, of the presence and the amount of active vincristine in human biological fluids.

It has been found that by using immunogens which are conjugates of an immunogenic polyamine polymer with a compound of the formula:

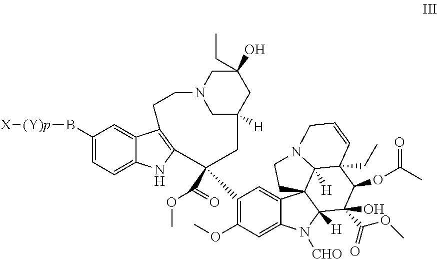

wherein Y, B, X and p are above;
wherein B is:

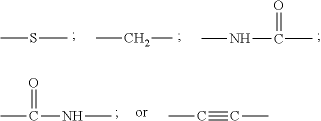

Y is an organic spacing group;
X is a terminal functional group capable of binding to a carrier; and
p is an integer from 0 to 1;
   produce antibodies which are specific for vincristine and do not substantially react with or bind to related pharmaceutically active vinca alkaloids, such as vinblastine, vindesine, and vinorelbine, as well as the pharmaceutically inactive metabolites of vincristine, such as M1.

The provision of these antibodies which substantially selectively react with vincristine and do not cross react with the pharmaceutically inactive metabolites of vincristine, such as M1 allows one to produce an immunoassay which can specifically detect and quantify so as to monitor vincristine in the fluid samples of patients being treated with vincristine. Also included within this invention are reagents and kits for said immunoassay.

### DETAILED DESCRIPTION

In accordance with this invention, a new class of antibodies is provided which is substantially selectively reactive to bind with vincristine and does not cross react to bind with the pharmacologically active vincristine related vinca alkaloids, such as vinblastine, vindesine, or vinorelbine, or with the pharmacologically inactive vincristine metabolites, particularly M1. It has been discovered that through the use of these derivatives of vincristine of formula III as immunogens, this new class of antibodies of this invention is provided. It is through the use of these antibodies that an immunoassay, including reagents and kits for such immunoassay for detecting and/or quantifying vincristine in blood, plasma or other body fluid samples has been developed. By use of this immunoassay, the presence and amount of vincristine in body fluid samples of patients being treated this therapeutic agent can be detected and/or quantified. In this manner, a patient being treated with vincristine can be monitored during therapy and his treatment adjusted in accordance with said monitoring. By means of this invention one achieves the therapeutic drug management of vincristine in cancer patients being treated with vincristine as therapeutic anti-neoplastic agent. The therapeutic anti-neoplastic agent to be detected is vincristine of formula I.

The immunoassay of this invention is carried out by providing a mixture containing the sample of the patient treated with vincristine and providing a mixture containing this sample with the antibody of this invention and the conjugate of a carrier of the ligand of formula III. In this manner, pharmaceutically active vincristine drug and the conjugate in said sample will bind with the antibody and from this binding, and from determining the amount of conjugate, one can calculate the amount of the drug in the patient's sample.

Any patient's sample can be used. Generally it is preferred that the patient sample can be a blood sample taken from the patient being treated with vincristine. This will provide an easy way to continuously monitor the treatment of the patient with this anti-neoplastic drug.

The reagents utilized in the assay of this invention are conjugates of a polymeric carrier with the compounds of formula III. These conjugates are competitive binding partners with the vincristine present in the sample for the binding with the antibodies of this invention. Therefore, the amount of conjugate reagent which binds to the antibody will be inversely proportional to the amount of vincristine in the sample. In accordance with this invention, the assay utilizes any conventional measuring means for detecting and measuring the amount of said conjugate which is bound or unbound to the antibody. Through the use of said means, the amount of the bound or unbound conjugate can be determined. Generally, the amount of vincristine in a sample is determined by correlating the measured amount of the bound or unbound conjugate produced by the vincristine in the sample with values of the bound or unbound conjugate determined from standard or calibration curve obtained with samples containing known amounts of vincristine, which known amounts are in the range expected for the sample to be tested. These studies for producing calibration curves are determined using the same immunoassay procedure as used for the sample.

### DEFINITIONS

Throughout this description the following definitions are to be understood:

The term “Ph” as used throughout this application designates a phenyl radical. The term “alkylene” designates a divalent saturated straight or branch chain hydrocarbon substituent containing from one to ten carbon atoms.

The terms “immunogen” and “immunogenic” refer to substances capable of eliciting, producing, or generating an immune response in an organism.

The term “conjugate” refers to any substance formed from the joining together of separate parts. Representative conjugates in accordance with the present invention include those formed by the joining together of a small molecule, such as the compound of formula III, and a large molecule, such as a carrier or a polyamine polymer, particularly protein. In the conjugate the small molecule maybe joined at one or more active sites on the large molecule. The term conjugate includes the term immunogen.

“Haptens” are partial or incomplete antigens. They are protein-free substances, mostly low molecular weight substances, which are not capable of stimulating antibody formation, but which do react with antibodies. The latter are formed by coupling a hapten to a high molecular weight immunogenic carrier and then injecting this coupled product, i.e., immunogen, into a human or animal subject. The hapten of this invention is vincristine.

As used herein, a “spacing group” or “spacer” refers to a portion of a chemical structure which connects two or more substructures such as haptens, carriers, immunogens, labels, or tracers through a functional linking group. These spacer groups will be enumerated hereinafter in this application. The atoms of a spacing group and the atoms of a chain within the spacing group are themselves connected by chemical bonds. Among the preferred spacers are straight or branched, saturated or unsaturated, carbon chains. Theses carbon chains may also include one or more heteroatoms within the chain or at termini of the chains. By “heteroatoms” is meant atoms other than carbon which are chosen from the group consisting of oxygen, nitrogen and sulfur. Spacing groups may also include cyclic or aromatic groups as part of the chain or as a substitution on one of the atoms in the chain.

The number of atoms in the spacing group is determined by counting the atoms other than hydrogen. The number of atoms in a chain within a spacing group is determined by counting the number of atoms other than hydrogen along the shortest route between the substructures being connected. A functional linking group may be used to activate, e.g., provide an available functional site on, a hapten or spacing group for synthesizing a conjugate of a hapten with a label or carrier or polyamine polymer.

An “immunogenic carrier,” as the terms are used herein, is an immunogenic substance, commonly a protein, that can join at one or more positions with a hapten, in this case vincristine, thereby enabling these hapten derivatives to induce an immune response and elicit the production of antibodies that can bind specifically with these haptens. The immunogenic carriers and the linking groups will be enumerated hereinafter in this application. Among the immunogenic carrier substances are included proteins, glycoproteins, complex polyamino-polysaccharides, particles, and nucleic acids that are recognized as foreign and thereby elicit an immunologic response from the host. The polyamino-polysaccharides may be prepared from polysaccharides using any of the conventional means known for this preparation.

Also various protein types may be employed as a poly(amino acid) immunogenic carrier. These types include albumins, serum proteins, lipoproteins, etc. Illustrative proteins include bovine serum albumin (BSA), keyhole limpet hemocyanin (KLH), egg ovalbumin, bovine thyroglobulin (BTG) etc. Alternatively, synthetic poly(amino acids) may be utilized.

Immunogenic carriers can also include poly amino-polysaccharides, which are a high molecular weight polymer built up by repeated condensations of monosaccharides. Examples of polysaccharides are starches, glycogen, cellulose, carbohydrate gums such as gum arabic, agar, and so forth. The polysaccharide also contains poly(amino acid) residues and/or lipid residues.

The immunogenic carrier can also be a poly(nucleic acid) either alone or conjugated to one of the above mentioned poly(amino acids) or polysaccharides.

The immunogenic carrier can also include solid particles. The particles are generally at least about 0.02 microns (μm) and not more than about 100 μm, and usually about 0.05 μM to 10 μm in diameter. The particle can be organic or inorganic, swellable or non-swellable, porous or non-porous, optimally of a density approximating water, generally from about 0.7 to 1.5 g/mL, and composed of material that can be transparent, partially transparent, or opaque. The particles can be biological materials such as cells and microorganisms, including non-limiting examples such as erythrocytes, leukocytes, lymphocytes, hybridomas, Streptococcus, Staphylococcus aureus, E. coli, and viruses. The particles can also be comprised of organic and inorganic polymers, liposomes, latex, phospholipid vesicles, or lipoproteins.

“Poly(amino acid)” or “polypeptide” is a polyamide formed from amino acids. Poly(amino acids) will generally range from about 2,000 molecular weight, having no upper molecular weight limit, normally being less than 10,000,000 and usually not more than about 600,000 daltons. There will usually be different ranges, depending on whether an immunogenic carrier or an enzyme is involved.

A “peptide” is any compound formed by the linkage of two or more amino acids by amide (peptide) bonds, usually a polymer of α-amino acids in which the α-amino group of each amino acid residue (except the NH₂ terminus) is linked to the α-carboxyl group of the next residue in a linear chain. The terms peptide, polypeptide and poly(amino acid) are used synonymously herein to refer to this class of compounds without restriction as to size. The largest members of this class are referred to as proteins.

A “label,” “detector molecule,” or “tracer” is any molecule which produces, or can be induced to produce, a detectable signal. The label can be conjugated to an analyte, immunogen, antibody, or to another molecule such as a receptor or a molecule that can bind to a receptor such as a ligand, particularly a hapten. Non-limiting examples of labels include radioactive isotopes, enzymes, enzyme fragments, enzyme substrates, enzyme inhibitors, coenzymes, catalysts, fluorophores, dyes, chemiluminescers, luminescers, or sensitizers; a non-magnetic or magnetic particle, a solid support, a liposome, a ligand, or a receptor.

The term “antibody” refers to a specific protein binding partner for an antigen and is any substance, or group of substances, which has a specific binding affinity for an antigen to the exclusion of other substances. The generic term antibody subsumes polyclonal antibodies, monoclonal antibodies and antibody fragments.

The term “derivative” refers to a chemical compound or molecule made from a parent compound by one or more chemical reactions.

The term “carrier” refers to solid particles and/or polymeric polymers such as immunogenic polymers such as those mentioned above. Where the carrier is a solid particle, the solid particle may be bound, coated with, or otherwise attached to a polyamine polymer to provide one or more reactive sites for bonding to the terminal functional group X in the compounds of the formula III.

The term “reagent kit,” or “test kit,” refers to an assembly of materials that are used in performing an assay. The reagents can be provided in packaged combination in the same or in separate containers, depending on their cross-reactivities and stabilities, and in liquid or in lyophilized form. The amounts and proportions of reagents provided in the kit can be selected so as to provide optimum results for a particular application. A reagent kit embodying features of the present invention comprises antibodies specific for vincristine. The kit may further comprise ligands of the analyte and calibration and control materials. The reagents may remain in liquid form or may be lyophilized.

The phrase “calibration and control materials” refers to any standard or reference material containing a known amount of a drug to be measured. The concentration of drug is calculated by comparing the results obtained for the unknown specimen with the results obtained for the standard. This is commonly done by constructing a calibration curve.

The term “biological sample” includes, but is not limited to, any quantity of a substance from a living thing or formerly living thing. Such living things include, but are not limited to, humans, mice, monkeys, rats, rabbits, horses, and other animals. Such substances include, but are not limited to, blood, serum, plasma, urine, cells, organs, tissues, bone, bone marrow, lymph, lymph nodes, synovial tissue, chondrocytes, synovial macrophages, endothelial cells, and skin.

### Reagents and Immunogens

In an immunoassay based upon an antibody, a conjugate of vincristine is constructed to compete with the vincristine in the sample for binding sites on the antibody. In the immunoassay of this invention, the reagents of formula III are the alkyl substituted vincristine derivatives formed on the 12′ position of vincristine of formula I. In the compounds of formula III the linker spacer constitutes the “Y-X” portion of this molecule. These linker X and the spacer Y are conventional in preparing conjugates for immunoassays and immunogens for producing antibodies. Any of the conventional spacer-linking groups utilized to prepare conjugates for immunoassays and immunogens for producing antibodies can be utilized in the compounds of formula III. Such conventional linkers and spacers are disclosed in U.S. Pat. No. 5,501,987 and U.S. Pat. No. 5,101,015.

The conjugates as well as the immunogens, are prepared from the compound of the formula III. In the conjugates or immunogens of the carrier with the hapten, the carriers are linked in one or more positions to one or more reactive amino groups contained by the polyamine polymer portion of the carrier to the hapten which has the formula:

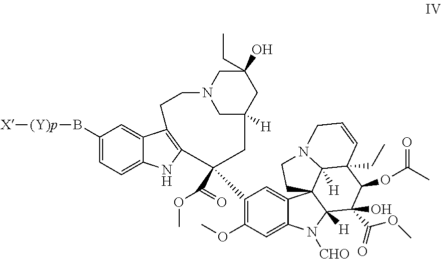

wherein X′ is —CH₂ or a functional linking group and p and Y are as above;

Among the preferred spacer groups are included the spacer groups hereinbefore mentioned. Particularly preferred spacing groups are groups such as alkylene containing from 1 to 10 carbon atoms,

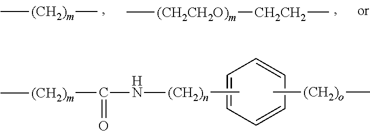

wherein n and o are integers from 0 to 6, and m is an integer from 1 to 10 with alkylene being the especially preferred spacing group.

In the compounds of formula IV, where X′ is a functional group linking the spacer, preferably through a reactive amine group on the polymeric carrier. The group X′ is the result of the terminal functional group X in the compounds of formula III binding to the reactive amino group in the polyamine polymer of the carrier or the immunogen. Any terminal functional group capable of reacting with an amino group can be utilized as the functional group X in the compounds of formula III. These terminal functional groups preferably included within X are:

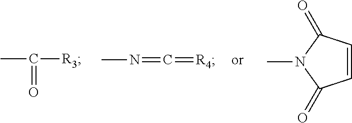

wherein R₃ is hydrogen, halogen, hydroxyl, or taken together with an attached oxygen atom forms a reactive ester, and R₄ is oxygen or sulfur. The radical —N═C=R₄ can be an isocyanate or an isothiocyanate. The active esters formed by R₃ include imidoester, such as N-hydroxysuccinamide, 1-hydroxy benzotriazole, pentafluorophenyl, and p-nitrophenyl ester. However any active ester which can react with an amine group can be used.

When X in the compound of formula III is

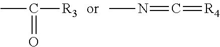

these compounds preferably react with the free amino group of the polymeric or immunogenic carrier. On the other hand, X in the compound of formula III can be the maleimide radical of the formula

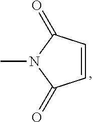

which reacts with both thiolate or thiol groups.

These maleimide compounds of formula III are reacted to attach to a polymeric protein which contains multiple thiols (such as thiolated dendrimers or dextrans), or polyamine carriers which have been modified to convert their amino groups to thiol groups. This can be done by the reacting a free amino group of a polymeric protein carrier with a compound of the formula

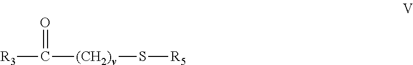

wherein R₅ is a thiol protecting group;
R₃ is as above; and
v is an integer of from 1 to 4.

This reaction is carried out in an aqueous medium by mixing the protein containing carrier with the compound of formula V in an aqueous medium. In this reaction temperature and pressure are not critical and the reaction can be carried out at room temperature and atmospheric pressure. Temperatures of from 10° C. to 25° C. are generally preferred. In the protein containing carrier which is reacted with the compound of formula V, any conventional thiol protecting agent can be utilized. The thiol protecting groups are well known in the art with 2-pyridyldthio being the preferred protecting group. By this reaction, the thiol group, —SH becomes the functional group of the carrier which bonds the compound of formula III to the remainder of the carrier.

Before reacting with the compound of formula V with the thiol modified carrier, the thiol protecting group of the thiol modified carrier is removed by conventional means. Any conventional means for removing a thiol protecting group can be utilized in carrying out this reaction. However, in utilizing a means to remove the thiol protecting group, care must be taken that the reactants be soluble in the aqueous medium and do not in any way destroy or harm the polyamine polymer contained in the carrier. A preferred means for removing this protecting group is by the use of dithiothreitol, tris-carboxyethyl phosphine (TCEP), and the like as agents to reduce the resultant condensation product. This reduction can be carried out by simply adding the reducing agent to the reaction medium without utilizing higher pressures or temperatures. This reduction can be carried out at room temperature and atmospheric pressure.

While the above method represents one means for converting a reactive terminal amino group on the polyamine polymeric containing carrier to a thiol group, any conventional means for carrying out this conversion can be utilized. Methods for converting terminal amino groups on polyamine polymeric containing carriers to thiol groups are well known in the art and can be employed in accordance with this invention.

The reaction of the polymeric polyamine containing carrier having a terminal reactive thiol group with the compound of formula V where X is a functional group capable of binding to the terminal thiol group carried by the carrier can be executed by conventional means. In this embodiment, the compound of formula V where X is maleimide is reacted with the thiol group carried by the polyamine polymeric carrier. Any well known means for addition of a thiol across a maleimide double bond can be utilized in producing the conjugates of formula IV which are conjugated to the carrier through a thiol bridge, where X′ is:

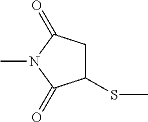

In the immunogens and conjugates of the preferred embodiment, the chemical bonds between the carboxyl group-containing hapten of formula III and the reactive amino groups on the polyamine polymer contained by the carrier or immunogen can be established using a variety of methods known to one skilled in the art. It is frequently preferable to form amide bonds. Amide bonds are formed by first activating the carboxylic acid moiety which forms X in the compounds of formula III and then reacting this carboxy group with a leaving group reagent (e.g., N-hydroxysuccinimide, 1-hydroxybenzotriazole, p-nitrophenol and the like). Any activating reagent such as dicyclohexylcarbodiimide, diisopropylcarbodiimide and the like can be used. The carboxylic acid moiety which forms X in the compounds of formula III can also be activated by conversion to the respective acid halides using thionyl chloride, thionyl bromide, and the like. The activated form of the carboxyl group in the vincristine hapten of formula III is then reacted with a buffered solution containing the carrier with the reactive amino group.

Where X, in the compounds of formula III contains an aldehyde radical, these compounds may be connected to the free amino group of the polyamine polypeptide on the carrier through an amine linkage by reductive amination. Any conventional method of condensing an aldehyde with an amine such as through reductive amination can be used to form this linkage. In this case, X′ in the ligand portions of formula IV is —CH₂—.

On the other hand where X is a terminal isocyanate or isothiocyanate radical —N═C═R₄, in the compound of formula III, these radicals when reacted with the free amine of a polyamine polymer to produce the conjugate or immunogen of formula IV where X′ is a urea or thiourea with the amino group on the polyamine carrier or the immunogenic polypeptide.

The compound of formula III can be converted into the immunogens and/or the conjugate reagents of this invention by reacting these compounds with a carrier containing a polyamine or a polypeptide. The same polypeptide can be utilized as the carrier and as the immunogenic polymer in the immunogen of this invention provided that polyamine or polypeptide is immunologically active. However, to form the conjugates, these polymers need not produce an immunological response as is needed for the immunogens. In accordance with this invention, the various functional group represented by X in the compounds of formula III can be conjugated to the carrier containing polymer with a reactive amino group by conventional means of attaching a functional group to an amino group contained within the polymer. In accordance with a preferred embodiment, in the compound of formula III, X is a carboxylic acid group or an active ester thereof.

In the preferred embodiment of the invention, the compounds of formula III can be formed from vincristine of formula I, where the 12′ position can first be selectively activated with iodoacetamide, to give the stable intermediate 12′-iodovincristine of formula VI:

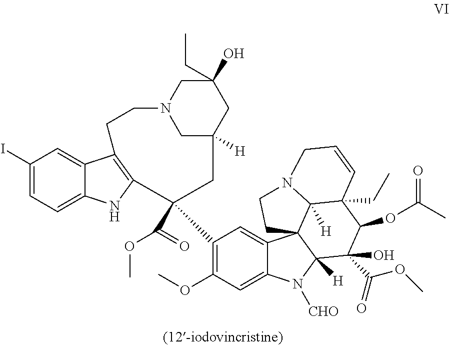

and then coupled to any alkyne of formula VII:

HC≡C—(Y)ₚ—X VII

The compounds of the formula III are formed from compounds of the formula VI and VII
Where p, Y and X are as above.

In forming these derivatives, any conventional means of reacting a terminal alkyne and halo-substituted aromatic ring to form a carbon-carbon bond can be utilized, although catalytic palladium is the most common means to condense the alkyne of formula VII with the 12′-iodo activated ring carbon on the compound of formula VI. On the other hand, were the compound of formula VII to contain functional groups which may interfere with this reaction to form these derivatives, these functional groups can be protected by means of suitable protecting groups which can be removed after this reaction by methods known to one skilled in the art.

Prior to this reaction, the functional group X on the compound of formula VII may be protected as described hereinabove with any conventional protecting group. These protecting groups can be removed after this condensation by conventional means known to one skilled in the art.

Compounds of formula III where B is —S— can be formed from the reaction of compound of formula VI with compounds of formula VIII

HS—(Y)ₚ—X VIII

Where p, Y and X are as above.

In forming these derivatives, any conventional means of reacting a primary thiol and halo-substituted aromatic ring to form a carbon-sulfur bond can be utilized, although catalytic palladium is the most common means to condense the thiol of formula VIII with the 12′-iodo activated ring carbon on the compound of formula VI. On the other hand, were the compound of formula VIII to contain functional groups which may interfere with this reaction to form these derivatives, these functional groups can be protected by means of suitable protecting groups which can be removed after this reaction by methods known to one skilled in the art.

Prior to this reaction, the functional group X on the compound of formula VIII may be protected as described hereinabove with any conventional protecting group. These protecting groups can be removed after this condensation by conventional means known to one skilled in the art.

Compounds of formula III where B is —CH₂— can be formed from the reaction of compound of formula VI with compounds of formula IX

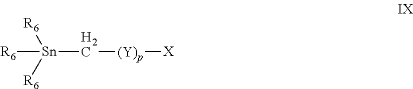

Where R₆ is any alkyl group and p, Y and X are as above.

In forming these derivatives, any conventional means of reacting a alkyl tin reagent and halo-substituted aromatic ring to form a carbon-sulfur bond can be utilized, although catalytic palladium under Stille coupling conditions is the most common means to condense the tin-liganded alkyl group of formula IX with the 12′-iodo activated ring carbon on the compound of formula VI. On the other hand, were the compound of formula IX to contain functional groups which may interfere with this reaction to form these derivatives, these functional groups can be protected by means of suitable protecting groups which can be removed after this reaction by methods known to one skilled in the art.

Prior to this reaction, the functional group X on the compound of formula IX may be protected as described hereinabove with any conventional protecting group. These protecting groups can be removed after this condensation by conventional means known to one skilled in the art.

Compounds of formula III where B is

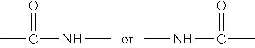

can be formed by first converting compound of formula VI to the corresponding carboxylic acid or amine by published protocols (Voss et al., 2009, Bioorganic & Medicinal Chemistry Letters) to give compound of formula X

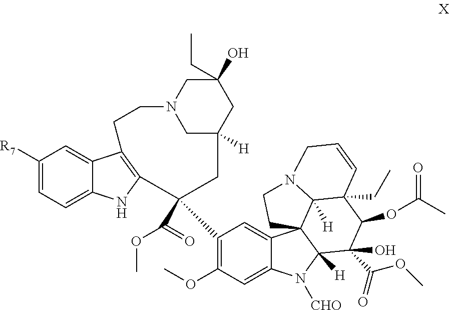

Where R₇ is either —NH₂ or —COOH,
and then reacting with compounds of formula XI

R₈—(Y)ₚ—X XI

Where R₈ is either —NH₂ or —COOH, and p, Y and X are as above.

In forming these derivatives, any conventional means of reacting an amine and carboxylic acid to form an amide bond can be utilized to condense the amine or carboxylic acid group of formula XI with the corresponding 12′ carboxylic acid or amine on the compound of formula X. On the other hand, were the compound of formula XI to contain functional groups which may interfere with this reaction to form these derivatives, these functional groups can be protected by means of suitable protecting groups which can be removed after this reaction by methods known to one skilled in the art.

Prior to this reaction, the functional group X on the compound of formula XI may be protected as described hereinabove with any conventional protecting group. These protecting groups can be removed after this condensation by conventional means known to one skilled in the art.

In preparing the amino bonded conjugates where the vincristine derivative of formula III contains a primary or secondary amino group as well as the carboxyl group, it may be necessary to use an amine protecting group during the activation and coupling reactions to prevent the conjugates from reacting with themselves. Typically, the amines on the vincristine derivative of formula III are protected by forming the corresponding N-trifluoroacetamide, N-tert-butyloxycarbonyl urethane (N-t-BOC urethane), N-carbobenzyloxy urethane or similar structure. Once the coupling reaction to the immunogenic polymer or carrier has been accomplished, as described above, the amine protecting group can be removed using reagents that do not otherwise alter the structure of the immunogen or conjugate. Such reagents and methods are known to one skilled in the art and include weak or strong aqueous or anhydrous acids, weak or strong aqueous or anhydrous bases, hydride-containing reagents such as sodium borohydride or sodium cyanoborohydride and catalytic hydrogenation.

Various methods of conjugating haptens and carriers are also disclosed in U.S. Pat. No. 3,996,344 and U.S. Pat. No. 4,016,146, which are herein incorporated by reference.

### Antibodies

The present invention also relates to novel antibodies including monoclonal antibodies to vincristine produced by utilizing the aforementioned immunogens. In accordance with this invention it has been found that these antibodies produced in accordance with this invention are selectively reactive with vincristine and do not react to bind with the related vinca alkaloids: vinblastine, vindesine, or vinorelbine, or to bind with the pharmacologically or pharmaceutically inactive vincristine metabolite M1 which may interfere with immunoassays for vincristine. The ability of the antibodies of this invention not to react with the pharmacologically or pharmaceutically inactive vincristine metabolite M1 makes these antibodies useful to provide an immunoassay for detecting the presence and/or quantifying the amount of vincristine in patient fluid samples.

The present invention relates to novel antibodies and monoclonal antibodies to vincristine. The antisera of the invention can be conveniently produced by immunizing host animals with the immunogens of this invention. Suitable host animals include rodents, such as, for example, mice, rats, rabbits, guinea pigs and the like, or higher mammals such as goats, sheep, horses and the like. Initial doses, bleedings and booster shots can be given according to accepted protocols for eliciting immune responses in animals, e.g., in a preferred embodiment mice received an initial dose of 100 μg immunogen/mouse, i.p. and two or more subsequent booster shots of between 50 and 100 μg immunogen/mouse over a six month period. Through periodic bleeding, the blood samples of the immunized mice were observed to develop an immune response against vincristine binding utilizing conventional immunoassays. These methods provide a convenient way to screen for hosts which are producing antisera having the desired activity.

The antibodies which are selectively reactive by binding with vincristine and have little substantial cross reactivity so as not bind to the pharmacologically or pharmaceutically inactive vincristine metabolite, M1, as well as bind to the pharmacologically active vincristine related vinca alkaloids: vinblastine, vindesine, or vinorelbine have a cross reactivity to these pharmacologically active vinca alkaloids and pharmaceutically inactive metabolites of less than 15%, preferably less than 5%, based on their reactivity with vincristine, can be produced utilizing the immunogen of formula III and by the screening method disclosed below. This screening method can be used to obtain antibodies which are reactive with vincristine chemotherapeutic agent, which are specific and selective bind to this chemotherapeutic agent, and do not have any relative reactivity, with regard to this chemotherapeutic agent, to bind to any related vinca alkaloid or any pharmacologically or pharmaceutically active or inactive vincristine metabolite such as M1.

In preparing these antibodies, an immunogenic carrier can be conjugated with the immunogen of formula III and used to immunize host animals such as mice, rabbits, sheep or rats. Development of the immune response to the compound of formula III can be monitored by ELISA utilizing microtiter plates coated with a conjugate of BSA and the compound of formula III. Once the immune response has been sufficiently developed the spleen cells of the host animal can be isolated and fused with an immortalized cell line. With respect to producing monoclonal antibodies, the fused cells can be plated on 96-well plates and grown in the presence of a selective medium to select hybridoma cells. Hybridoma supernatants and antisera can be assayed for the presence of anti-vincristine antibodies by ELISA. Antibodies from wells that gave positive ELISA results can be tested for vincristine binding by indirect competitive microtiter plate assay. The IC₅₀ values of an analyte such as vincristine, vinblastine, or its metabolite M1, can be calculated from this assay. The IC₅₀ (inhibitory concentration at 50%) of an analyte in an assay is the concentration of that analyte in a sample at which the signal in the assay is 50% of the total signal for the assay in the absence of analyte in an inhibition assay. Selective reactivity of an analyte is calculated from a ratio of the IC₅₀'s expressed as a %: 100%−([IC₅₀-analyte/(IC₅₀-vincristine+IC₅₀-analyte)]×100). The calculation of the IC₅₀ is carried out according to the procedure found in The Immunoassay Handbook, pp 108-110, 3^{rd} edition, edited by D. Wild, published by Elsevier, Amsterdam, 2005. As seen from the formula, the IC₅₀ of an analyte is inversely proportional to the reactivity of the analyte. Cells from wells that gave 100% or approaching 100% can be subcloned by limiting dilution to isolate individual clones producing monoclonal anti-vincristine antibodies. Cells from wells that have desired relative reactivity with regard to vincristine can be subcloned by limiting dilution to isolate individual clones producing monoclonal anti-vincristine antibodies.

Monoclonal antibodies are produced conveniently by immunizing Balb/c mice according to the above schedule followed by injecting the mice with 100 μg immunogen i.p. or i.v. on three successive days starting three days prior to the cell fusion. Other protocols well known in the antibody art may of course be utilized as well. The complete immunization protocol detailed herein provided an optimum protocol for serum antibody response for the antibody to vincristine.

B lymphocytes obtained from the spleen, peripheral blood, lymph nodes or other tissue of the host may be used as the monoclonal antibody producing cell. Most preferred are B lymphocytes obtained from the spleen. Hybridomas capable of generating the desired monoclonal antibodies of the invention are obtained by fusing such B lymphocytes with an immortal cell line, which is a cell line that which imparts long term tissue culture stability on the hybrid cell. In the preferred embodiment of the invention the immortal cell may be a lymphoblastoid cell or a plasmacytoma cell such as a myeloma cell, itself an antibody producing cell but also malignant. Murine hybridomas which produce monoclonal antibodies to vincristine are formed by the fusion of mouse myeloma cells and spleen cells from mice immunized against vincristine-protein conjugates. Chimeric and humanized monoclonal antibodies can be produced by cloning the antibody expressing genes from the hybridoma cells and employing recombinant DNA methods now well known in the art to either join the subsequence of the mouse variable region to human constant regions or to combine human framework regions with complementary determining regions (CDR's) from a donor mouse or rat immunoglobulin. An improved method for carrying out humanization of murine monoclonal antibodies which provides antibodies of enhanced affinities is set forth in International Patent Application WO 92/11018.

Polypeptide fragments comprising only a portion of the primary antibody structure may be produced, which fragments possess one or more immunoglobulin activities. These polypeptide fragments may be produced by proteolytic cleavage of intact antibodies by methods well known in the art, or by inserting stop codons at the desired locations in expression vectors containing the antibody genes using site-directed mutageneses to produce Fab fragments or (Fab′)₂ fragments. Single chain antibodies may be produced by joining VL and VH regions with a DNA linker (see Huston et al., Proc. Natl. Acad. Sci. U.S.A., 85:5879-5883 (1988) and Bird et al., Science, 242:423-426 (1988))

The antibodies of this invention are selective for binding to vincristine without having any substantial cross-reactivity to bind with the pharmacologically active vincristine related vinca alkaloids, such as vinblastine, vindesine, or vinorelbine, or to bind with the pharmacologically inactive vincristine metabolites, particularly M1. By having no substantial cross-reactivities, it is meant that the antibodies of this invention have a cross reactivity, relative to their reactivity with vincristine, to bind with the pharmacologically related vinca alkaloids vinblastine, vindesine, vinorelbine as well as to bind with the pharmaceutically inactive vincristine metabolites, particularly M1, of less than 15%, preferably less than 5%.

### Immunoassays

In accordance with this invention, the conjugates and the antibodies generated from the immunogens of these compounds of formula III can be utilized as reagents for the determination of vincristine in patient samples. This determination is performed by means of an immunoassay. Any immunoassay in which the reagent conjugates formed from the compounds of formula III compete with the vincristine in the sample for binding sites on the antibodies generated in accordance with this invention can be utilized to determine the presence of vincristine in a patient sample. The manner for conducting such an assay for vincristine in a sample suspected of containing vincristine, comprises combining an (a) aqueous medium sample, (b) an antibody to vincristine generated in accordance with this invention and (c) the conjugates formed from the compounds of formula III or mixtures thereof. The amount of vincristine in the sample can be determined by measuring the inhibition of the binding to the specific antibody of a known amount of the conjugate added to the mixture of the sample and antibody. The result of the inhibition of such binding of the known amount of conjugates by the unknown sample is compared to the results obtained in the same assay by utilizing known standard solutions of vincristine. In determining the amount of vincristine in an unknown sample, the sample, the conjugates formed from the compounds of formula III and the antibody may be added in any order.

Various means can be utilized to measure the amount of conjugate formed from the compounds of formula III bound to the antibody. One method is where binding of the conjugates to the antibody causes a decrease in the rate of rotation of a fluorophore conjugate. The amount of decrease in the rate of rotation of a fluorophore conjugate in the liquid mixture can be detected by the fluorescent polarization technique such as disclosed in U.S. Pat. No. 4,269,511 and U.S. Pat. No. 4,420,568.

On the other hand, the antibody can be coated or absorbed on nanoparticles so that when these particles react with the vincristine conjugates formed from the compounds of formula IV, these nanoparticles form an aggregate. However, when the antibody coated or absorbed nanoparticles react with the vincristine in the sample, the vincristine from the sample bound to these nanoparticles does not cause aggregation of the antibody nanoparticles. The amount of aggregation or agglutination can be measured in the assay mixture by absorbance.

On the other hand, these assays can be carried out by having either the antibody or the vincristine conjugates attached to a solid support such as a microtiter plate or any other conventional solid support including solid particles. Attaching antibodies and proteins to such solid particles is well known in the art. Any conventional method can be utilized for carrying out such attachments. In many cases, in order to aid measurement, labels may be placed upon the antibodies, conjugates or solid particles, such as radioactive labels or enzyme labels, as aids in detecting the amount of the conjugates formed from the compounds of formula III which is bound or unbound with the antibody. Other suitable labels include chromophores, fluorophores, etc.

As a matter of convenience, assay components of the present invention can be provided in a kit, a packaged combination with predetermined amounts of new reagents employed in assaying for vincristine. These reagents include the antibody of this invention, as well as, the conjugates formed from the compounds of formula IV.

In addition to these necessary reagents, additives such as ancillary reagents may be included, for example, stabilizers, buffers and the like. The relative amounts of the various reagents may vary widely to provide for concentrations in solution of the reagents which substantially optimize the sensitivity of the assay. Reagents can be provided in solution or as a dry powder, usually lyophilized, including excipients which on dissolution will provide for a reagent solution having the appropriate concentrations for performing the assay.

### EXAMPLES

In the examples, the following abbreviations are used for designating the following:
TFA trifluoroacetic acid
DMF Dimethylformamide
DMSO Dimethylsulfoxide
s-NHS sulfo-N-hydroxy succinimide
EDC 1-(3-dimethylaminopropyl)-3-ethylcarbodiimide hydrochloride
KLH Keyhole Limpet Hemocyanin
BSA Bovine serum albumin
PBS Phosphate buffered saline
NaCl sodium chloride
HRP horse radish-peroxidase
ANS 8-Anilino-1-naphthalenesulfonic acid
TMB 3,3′,5,5′-Tetramethylbenzidine
TRIS Tris(hydroxymethyl)aminomethane hydrochloride
diH₂O deionized water

The phosphate buffer composition has an aqueous solution containing
15.4 mM Sodium phosphate dibasic (Na₂HPO₄)
4.6 mM Sodium phosphate monobasic (NaH₂PO₄)
pH=7.2±0.10

In the examples, Scheme 1 below sets forth the specific compounds prepared and referred to by numbers in the Examples. The scheme is as follows:

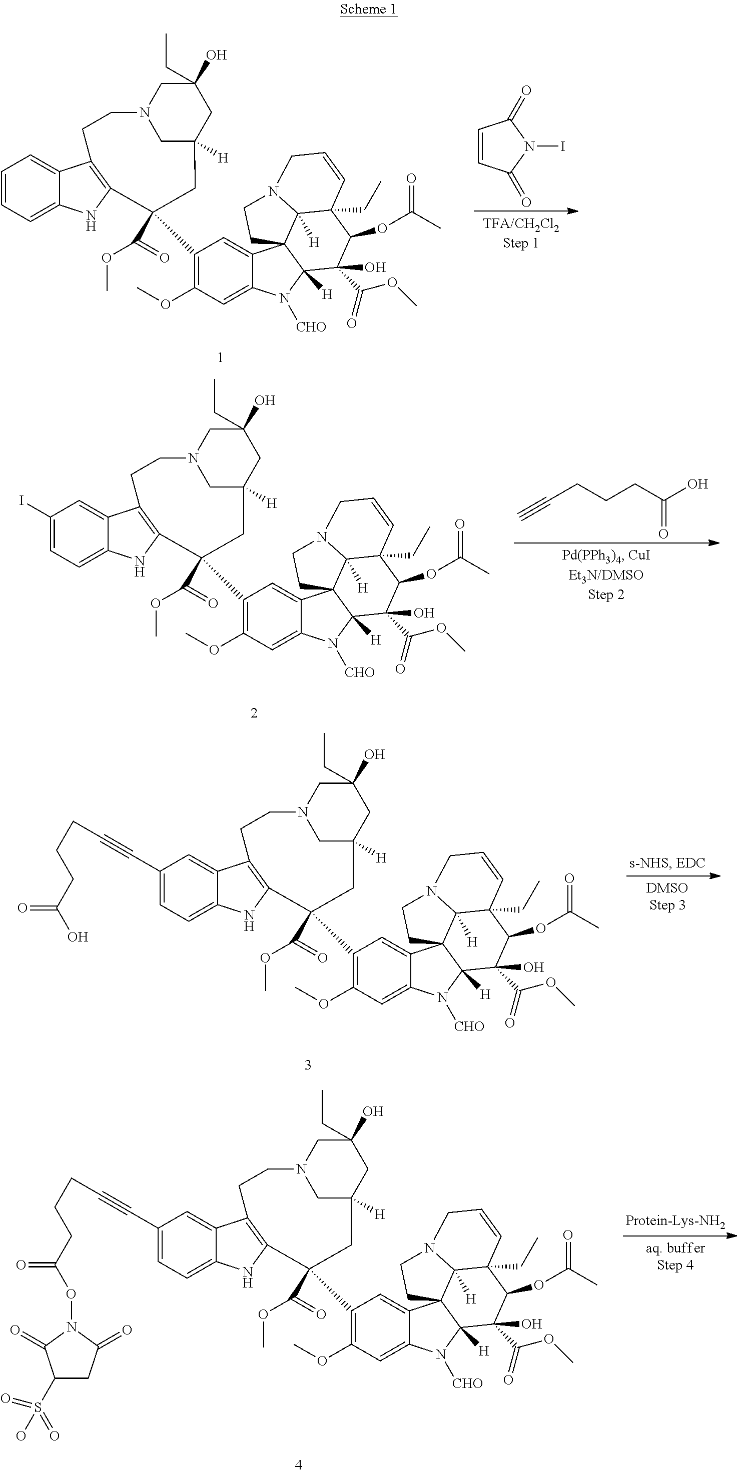

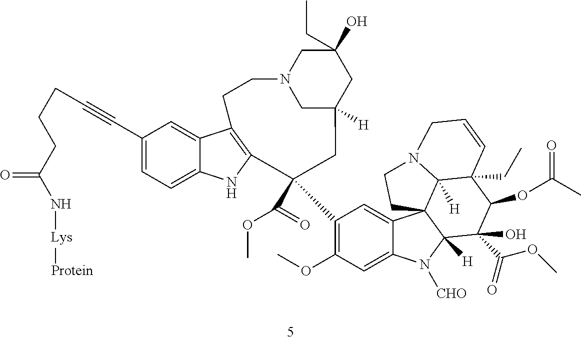

### Example 1

### Preparation of 12′-(2-carboxy-5-hexynyl)vincristine derivative [3] (Scheme 1)

Vincristine hydrogensulfate [1] (200 mg, 0.217 mmole) was dissolved in 8 mL of a mixture of trifluoracetic acid/methylene chloride (1:1) and maintained at −15° C. While stirring the cold vincristine solution, a 5 mL solution of N-iodosuccinimide (48.5 mg, 0.217 mmol) in trifluoroacetic acid/methylene chloride (1:1), cooled to 0° C., was added dropwise over 30 minutes by addition funnel. The reaction mixture was stirred an additional 30 minutes after N-iodosuccinimide addition. The reaction mixture was then treated with saturated aqueous sodium bicarbonate, and further neutralized with sodium hydroxide (3 N) until a pH of 8 was obtained. The neutralized solution was diluted with 150 mL of methylene chloride, and the resultant organic layer was washed with water and then brine, and subsequently dried over Na₂SO₄. The organic solvent was removed by rotary evaporation to provide 12′-iodovincristine [2] (182 mg, yield 89%) as a tan powder which was carried forward without further purification.

The intermediate 12′-iodovincristine [2] (1.0 eq) was dissolved in dimethylsulfoxide/triethylamine (1:1) along with 5-hexynoic acid (1.1 eq), CuI (0.09 eq), and Pd(PPh₃)₄ (6 mol %, 0.06 eq). The resulting mixture was irradiated under microwave conditions at 60° C. for 1-2 hours. After irradiation, the mixture was filtered through a Celite pad. The filtrate and several dichloromethane washes of the Celite were combined and concentrated by rotary evaporation. The final product [3] in the concentrated dimethylsulfoxide solution was purified by reversed-phase flash chromatography using 0.1% formate/acetonitrile and 1% formate/water as eluent. Eluted fractions containing pure product were combined, rotary evaporated to remove excess acetonitrile, and then lyophilized to isolate the desired compound 12′-(2-carboxy-5-hexynyl)vincristine [3] as a yellow solid (yield 42%).

### Example 2

General Method for Preparing s-NHS Activated Drug Derivatives from the Corresponding Acid [3]

Vincristine derivative [3] was activated with EDC and s-NHS to produce the s-NHS activated ester of vincristine [4] for eventual conjugation to proteins (examples 4 and 5).

### Example 3

### Preparation of s-NHS Activated ester 12′-(2-carboxy-5-hexynyl)vincristine acid derivative [4]

Vincristine derivative [3], example 1, scheme 1, (92.9 mg) was dissolved in 10.5 mL of DMSO to which was added s-NHS (49.6 mg) and EDC (56.1 mg). The reaction mixture was stirred for 20 hours at ambient temperature under a nitrogen atmosphere to produce the s-NHS activated ester of vincristine [4]. The reaction mixture was used directly in examples 4 and 5.

### Example 4

### Preparation of KLH Immunogen with Activated Hapten [4]

A protein solution of KLH was prepared by dissolving 200 mg of KLH in 18 mL of phosphate buffer (50 mM, pH 7.5), followed by addition of 6.15 mL of s-NHS activated vincristine derivative [4] prepared in Example 3. The reaction mixture of KLH and activated vincristine derivative [4] was allowed to stir for 20 hours at room temperature to produce the vincristine [5]-KLH conjugate. The vincristine [5]-KLH conjugate was then purified by dialysis against 30% DMSO in phosphate buffer (50 mM, pH 7.5) at room temperature. Thereafter the DMSO proportion was reduced stepwise: 25%, 10% and 0%. The last dialysis was performed against phosphate buffer at 4° C. The vincristine [5]-KLH conjugate was characterized by ultraviolet-visible spectroscopy. The conjugate was diluted to a final concentration of 2 mg/mL in phosphate buffer (50 mM, pH 7.5).

### Example 5

### Preparation of BSA Conjugate with Activated Hapten [4]

A protein solution of BSA was prepared by dissolving 1 g BSA in phosphate buffer (50 mM, pH 7.5) for a final concentration of 50 mg/mL. To this protein solution was added 1.57 mL of s-NHS activated vincristine derivative [4] prepared in Example 3. The amount of s-NHS activated vincristine derivative [4] added to the protein solution of BSA was calculated for a 1:1 molar ratio between the derivative of vincristine [4] and BSA. The mixture of BSA and activated vincristine derivative [4] was allowed to stir for 18 hours at room temperature to produce the conjugate of the activated vincristine ester [4] and BSA. This conjugate was then purified by dialysis against 20% DMSO in phosphate buffer (50 mM, pH 7.5) at room temperature. Thereafter the DMSO proportion was reduced stepwise: 10% and 0%. The last dialysis was performed against phosphate buffer at 4° C. The purified vincristine [5]-BSA conjugate was characterized by UV/VIS spectroscopy.

### Example 6a

### Preparation of Polyclonal Antibodies to Vincristine [5]

Ten female BALB/c mice were immunized i.p. with 100 μg/mouse of vincristine [5]-KLH immunogen, as prepared in Example 4, emulsified in Complete Freund's adjuvant. The mice were boosted once, four weeks after the initial injection with 100 μg/mouse of the same immunogen emulsified in Incomplete Freund's Adjuvant. Twenty days after the boost, test bleeds containing polyclonal antibodies from each mouse were obtained by orbital bleed. The test bleeds were fractionated by centrifugation to yield anti-sera. The anti-sera from these test bleeds, which contain polyclonal antibodies to vincristine [5]-KLH immunogen were evaluated in Examples 8 and 9.

### Example 6b

### Preparation of Monoclonal Antibodies to Vincristine [5]

Mice from example 6a that were immunized with vincristine [5]-KLH prepared in Example 4 were used to produce monoclonal antibodies. For monoclonal antibodies starting three days before the fusion, the mice were injected i.p. with 400 μg (3 days before fusion), 200 μg (2 days before fusion), and 200 μg (1 day before fusion) of vincristine [5]-KLH in PBS/DMSO prepared in Example 4. Spleen cells were isolated from the selected mice and fused with 2×10⁷ SP2/0 cells with 50% polyethylene glycol 1500 according to the method of Coligan, J. E. et al., eds., Current Protocols in Immunology, 2.5.1-2.5.8, (1992), Wiley & Sons, NY. The fused cells were plated on ten 96-well plates in DMEM/F12 supplemented with 20% FetalClone I, 2% L-glutamine (100 mM) and 2% 50×HAT. Two to three weeks later, the hybridoma supernatant was assayed for the presence of anti-vincristine antibodies by ELISA (as in example 8b). Cells from the wells that gave positive ELISA results (example 8b) were expanded to 24 well plates. Clones positive by ELISA were subcloned twice by limiting dilution according to the method disclosed in Coligan, J. E. et al., eds., Current Protocols in Immunology, 2.5.8-2.5.17, (1992), Wiley & Sons, NY. Hybridoma culture supernatants containing monoclonal antibody from selected subclones were confirmed for vincristine binding by a competitive ELISA (example 9).

### Example 7

Microtiter Plate Sensitization Procedure with Vincristine [5]-BSA Conjugate

The ELISA method for measuring vincristine concentrations was performed in polystyrene microtiter plates (Nunc MaxiSorp F8 Immunomodules) optimized for protein binding and containing 96 wells per plate. Each well was coated with vincristine [5]-BSA conjugate (prepared as in Example 5) by adding 300 μl, of vincristine [5]-BSA conjugate at 10 μg/mL in 0.05M sodium carbonate, pH 9.6, and incubating for three hours at room temperature. The wells were washed with 0.05M sodium carbonate, pH 9.6 and then were blocked with 375 μL of 5% sucrose, 0.2% sodium caseinate solution for 30 minutes at room temperature. After removal of the blocking solution the plates were dried at 37° C. overnight.

### Example 8a

### Antibody Screening Procedure—Titer

This procedure is to find the dilution of antibody or anti-serum to be tested for displacement as in Example 9. The ELISA method for screening vincristine antibodies (produced in Example 6) was performed with the microtiter plates that were sensitized with vincristine-BSA conjugate prepared in Example 7. The antibody screening assay was performed by diluting the murine serum from test bleeds (as in Example 6a) containing polyclonal vincristine antibodies to 1:2,000, 1:6,000, 1:18,000 and 1:54,000 (volume/volume) in phosphate buffered saline containing 0.1% BSA and 0.01% thimerosal. For evaluation of monoclonal antibodies, hybridoma supernatants of Example 6b, which were found to be positive for the presence of antibody by the procedure of Example 8b were diluted 1:2, 1:4, 1:16, etc. (volume/volume) in phosphate buffered saline containing 0.1% BSA and 0.01% thimerosal. To each well of vincristine-BSA sensitized wells (prepared in Example 7)50 μL phosphate buffered saline containing 0.1% BSA and 0.01% thimerosal and 50 μL of diluted antibody were added and incubated for 10 minutes at room temperature with shaking. During this incubation antibody binds to the vincristine-BSA conjugate passively absorbed in the wells (Example 7). The wells of the plates were washed three times with 0.02 M TRIS, 0.9% NaCl, 0.5% Tween-80 and 0.001% thimerosal, pH 7.8 to remove any unbound antibody. To detect the amount of vincristine antibody bound to the vincristine[5]-BSA conjugate in the wells, 100 μL of a goat anti-mouse antibody-HRP enzyme conjugate (Jackson Immunoresearch) diluted to a specific activity (approximately 1/3000) in PBS with 0.1% BSA, 0.05% ANS, 0.01% thimerosal, capable of binding specifically with murine immunoglobulins and producing a colored product when incubated with a substrate, in this example TMB, were added to each well. After an incubation of 10 minutes at room temperature with shaking, during which the goat anti-mouse antibody-HRP enzyme conjugate binds to vincristine antibodies in the wells, the plates were again washed three times to remove unbound goat anti-mouse antibody-HRP enzyme conjugate. To develop a measurable color in the wells washing was followed by the addition of 100 μL of TMB (TMB Substrate, BioFx), the substrate for HRP, to develop color during a 10 minute incubation with shaking at room temperature. Following the incubation for color development, 50 μL of stop solution (1.5% sodium fluoride in diH₂O) was added to each well to stop the color development and after 10 seconds of shaking the absorbance was determined at 650 nm (Molecular Devices Plate Reader). The amount of antibody in a well was proportional to the absorbance measured and was expressed as the dilution (titer) resulting in an absorbance of 1.5. Titers were determined by graphing antibody dilution of the antibody measured (x-axis) vs. absorbance 650 nm (y-axis) and interpolating the titer at an absorbance of 1.5. The titer which produced absorbance of 1.5 determined the concentration (dilution) of antibody used in the indirect competitive microtiter plate assay described in Example 9.

### Example 8b

### Antibody Screening Procedure—Monoclonal Screening

The ELISA method for screening vincristine monoclonal antibodies (produced in Example 6b) was performed with the microtiter plates that were sensitized with vincristine[5]-BSA as described in example 7. To each well of vincristine[5]-BSA sensitized wells (prepared in example 7) 50 μL phosphate buffered saline containing 0.1% BSA and 0.01% thimerosal and then 50 μL of monoclonal culture supernatant were added and incubated for 10 minutes at room temperature with shaking. During this incubation antibody binds to the vincristine-conjugate in the well. The wells of the plates were washed three times with 0.02 M TRIS, 0.9% NaCl, 0.5% Tween-80 and 0.001% thimerosal, pH 7.8 to remove any unbound antibody. To detect the amount of vincristine antibody bound to the vincristine[5]-BSA conjugate in the wells, 100 μL of a goat anti-mouse antibody-HRP enzyme conjugate (Jackson Immunoresearch) diluted 1/3000 in PBS with 0.1% BSA, 0.05% ANS, 0.01% thimerosal, capable of binding specifically with murine immunoglobulins and producing a colored product when incubated with a substrate, in this example TMB, were added to each well. After an incubation of 10 minutes at room temperature with shaking, during which the goat anti-mouse antibody-HRP enzyme conjugate binds to vincristine antibodies in the wells, the plates were again washed three times to remove unbound goat anti-mouse antibody-HRP enzyme conjugate. To develop a measurable color in the wells washing was followed by the addition of 100 μL of TMB (TMB Substrate, BioFx), the substrate for HRP, to develop color during a 10 minute incubation with shaking at room temperature. Following the incubation for color development, 50 μL of stop solution (1.5% sodium fluoride in di H₂O) was added to each well to stop the color development and after 10 seconds of shaking the absorbance was determined at 650 nm (Molecular Devices Plate Reader). The amount of antibody in a well was proportional to the absorbance measured. Samples with an absorbance of greater than three or more times background were designated as positive. Fifty samples with highest absorbance were expanded to 24 well plates, as described in Example 6b.

### Example 9

Indirect Competitive Microtiter Plate Immunoassay Procedure Determining IC₅₀ and Cross-Reactivity for Antibodies to Vincristine

The ELISA method for determining IC₅₀ values and cross-reactivity was performed with the microtiter plates that were sensitized with vincristine [5]-BSA conjugates as described in Example 7. The analytes, vincristine and vinblastine were diluted in diH₂O over a concentration range of 1 to 10,000 ng/mL when using vincristine [5]-BSA microtiter plates (as in Example 7). Each of the assays were performed by incubating 50 μL of the analyte solution with 50 μL of one of the anti-sera selected from the polyclonal antibodies produced in Example 6a with the immunogen of Example 4 or 50 μL of one of the selected monoclonal antibodies produced in Example 6b. The assays were all performed by diluting the concentration of the anti-sera or monoclonal antibodies in each of the wells to the titer determined in Example 8a. During the 10 minute incubation (at room temperature with shaking) there is a competition of antibody binding for the vincristine [5]-BSA conjugate in the well (produced in Example 7) and the analyte in solution. Following this incubation the wells of the plate were washed three times with 0.02 M TRIS, 0.9% NaCl, 0.5% Tween-80 and 0.001% thimerosal, pH 7.8 to remove any material that was not bound. To detect the amount of vincristine antibody bound to the vincristine [5]-BSA conjugate in the wells (produced in Example 7), 100 μL of a goat anti-mouse antibody-HRP enzyme conjugate (Jackson Immunoresearch) diluted to a predetermined specific activity (approximately 1/3000) in PBS with 0.1% BSA, 0.05% ANS, 0.01% thimerosal, capable of binding specifically with murine immunoglobulins and producing a colored product when incubated with a substrate, in this example TMB, were added to each well. After an incubation of 10 minutes at room temperature with shaking, during which the goat anti-mouse antibody-HRP enzyme conjugate binds to vincristine antibodies in the wells, the plates were again washed three times to remove unbound secondary conjugate. To develop a measurable color in the wells washing was followed by the addition of 100 μl, of TMB (TMB Substrate, BioFx), the substrate for HRP, to develop color in a 10 minute incubation with shaking at room temperature. Following the incubation for color development, 50 μL of stop solution (1.5% sodium fluoride in di H₂O) was added to each well to stop the color development and after 10 seconds of shaking the absorbance was determined at 650 nm (Molecular Devices Plate Reader). The amount of antibody in a well was proportional to the absorbance measured and inversely proportional to the amount of analyte in the sample. The IC₅₀'s of vincristine and vinblastine were determined by constructing dose-response curves with the absorbance in the wells plotted versus analyte concentration in the wells. The absorbance of the color in the wells containing analyte was compared to that with no analyte and a standard curve was generated. The IC₅₀ value for a given analyte was defined as the concentration of analyte that was required to have 50% of the absorbance of the wells containing no analyte. The cross-reactivity was calculated as the ratio of the IC₅₀ for vincristine to the IC₅₀ for vinblastine and expressed as a percent. When measured with this pool of antibodies, the percent cross-reactivities relative to vincristine for vinblastine were less than 5%. Results for polyclonal antibodies to vincristine are in table I below. Results for monoclonal antibodies to vincristine are in table II.

TABLE I
Cross-reactivity of competitive immunoassay using
polyclonal antibodies to vincristine (Example 6a).

| | Bleed # | G1M1 | G1M3 | G2M1 | G2M5 | G1M4 |
|---|---|---|---|---|---|---|
| Analyte | Vincristine | 100% | 100% | 100% | 100% | 100% |
| | Vinblastine | <0.9% | <3.9% | <3.4% | <2% | <4.1% |

TABLE II
Cross-reactivity of competitive immunoassay using monoclonal
antibodies to vincristine (Example 6b).

Monoclonal antibody
number

| Analyte | 1D12.10 | 8B6.23.23 |
|---|---|---|
| Vincristine | 100% | 100% |
| Vinblastine | <0.1% | 0.3% |
| Vindesine | <0.1% | <0.1% |
| Vinorelbine | <0.1% | 0.7% |

As seen from these tables, the antibodies of this invention are substantially selectively reactive with the active form of vincristine as well as substantially non-cross-reactive with related vinca alkaloids vinblastine (IIA), vindesine (JIB), and vinorelbine (IIC).

What is claimed:

1. An antibody which bonds to selectively binds to vincristine and does not substantiate cross react by binding with the pharmaceutically inactive vincristine metabolite, M1.

2. The antibody of claim 1 wherein said selectively reactive antibody, selectively binds to vincristine without any substantial cross reactivity to bind with vinblastine, vindesine, or vinorelbine, and with the pharmaceutically inactive vincristine metabolite, M1.

3. The antibody of claim i wherein said antibody is generated from an immunogen comprising an immunogenic carrier containing a polyamine polymer conjugated with a ligand of the formula: 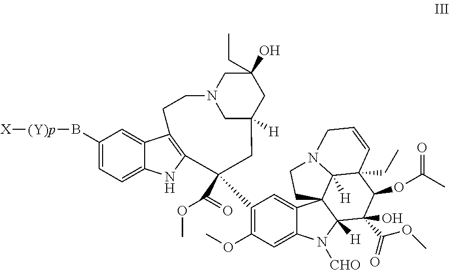
wherein B is 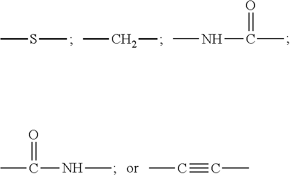
Y is an organic spacing group;
X is a terminal functional group capable of binding to an immunogenic carrier containing a polyamine polymer; and
p is an integer from 0 to 1

4. The antibody of claim 14, wherein said antibody is derived from mice, sheep, rabbits or rats.

5. The antibody of claim 3, wherein said antibody is a monoclonal antibody.

6. A compound of the formula: 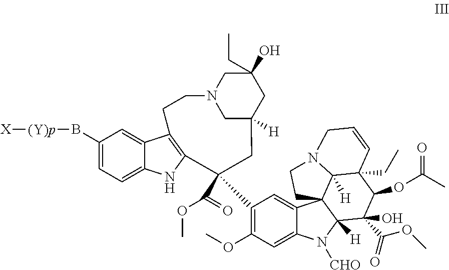
wherein B is 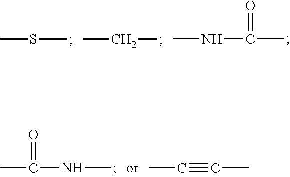
Y is an organic spacing group;
X is a terminal functional group capable of binding to a carrier; and
p is an integer from 0 to 1.

7. The compound of claim 6, wherein p is 0.

8. The compound of claim 7, wherein X is 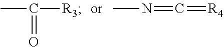
wherein R₃ is hydrogen, a halide, a hydroxyl, or taken together with its attached oxygen atom forms a reactive ester and R₄ is oxygen or sulfur.

9. The compound of claim 8 wherein X is

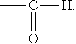

10. The compound of claim 8 wherein X is

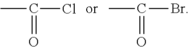

11. The compound of claim 8, wherein X is 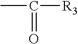
and R₃ is hydroxyl.

12. The compound of claim 8, wherein X is 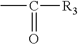
and R₃ forms a reactive ester.

13. The compound of claim 12, wherein the ester formed is a lower alkyl ester, imidoester or amidoester.

14. The compound of claim 6, wherein p is 1.

15. The compound of claim 14, wherein X is 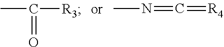
wherein R₃ is hydrogen, a halide, a hydroxyl, or taken together with its attached oxygen atom forms a reactive ester and R₄ is oxygen or sulfur.

16. The compound of claim 15, wherein Y is alkylene containing from 1 to 10 carbon atoms, 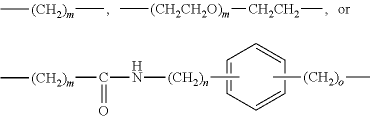
wherein n and o are integers from 0 to 6, and m is an integer from 1 to 6.

17. The compound of claim 16, wherein Y is lower alkylene containing from 1 to 6 carbon atoms.

18. The compound of claim 17, wherein X is 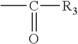
and R₃ is as above.

19. The compound of claim 16, wherein Y is 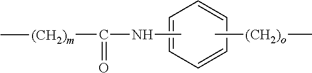
wherein m and o are as above.

20. The compound of claim 19, wherein X is 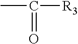
and R₃ is as above.

21. A conjugate comprising a carrier conjugated with a ligand of the formula: 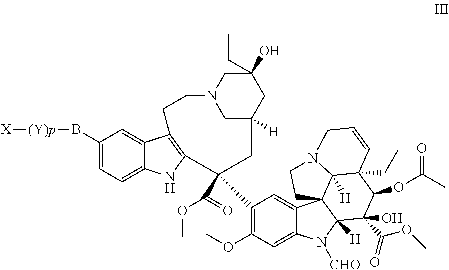
wherein B is 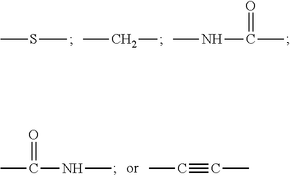
Y is an organic spacing group;
X is a functional linking group capable of linking to a carrier; and
p is an integer from 0 to 1.

22. The conjugate of claim 21, wherein the carrier contains a polyamine polymer.

23. The conjugate of claim 21, wherein p is 0.

24. The conjugate of claim 23, wherein X is 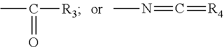
wherein R₃ is hydrogen, a halide, a hydroxyl, or taken together with its attached oxygen atom forms a reactive ester and R₄ is oxygen or sulfur.

25. The conjugate of claim 21, wherein p is 1.

26. The conjugate of claim 25, wherein Y is alkylene containing from 1 to 10 carbon atoms, 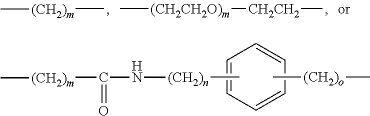
wherein n and o are integers from 0 to 6, and m is an integer from 1 to 6.

27. The conjugate of claim 26, wherein X is 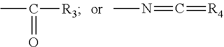
wherein R₃ is hydrogen, a halide, a hydroxyl, or taken together with its attached oxygen atom forms a reactive ester and R₄ is oxygen or sulfur.

28. The conjugate of claim 21, wherein said polyamine polymer is an immunogenic polymer.

29. The conjugate of claim 26 wherein Y is an alkylene containing from 1 to 6 carbon atoms.

30. The conjugate of claim 29, wherein X is 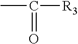
and R₃ is as above.

31. The conjugate of claim 26, wherein Y is 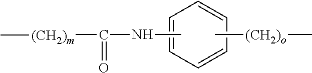
where m and o are as above.

32. The conjugate of claim 31, wherein X is 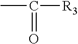
and R₃ is as above.
